(19) United States
(12) Patent Application Publication
(10) Pub. No.: US 2023/0372160 A1
(43) Pub. Date: Nov. 23, 2023

(54) VENTILATION SYSTEM

(71) Applicant: VTVP LLC, Dallas, TX (US)

(72) Inventor: Hunter Cole RYAN, Dallas, TX (US)

(21) Appl. No.: 18/200,336

(22) Filed: May 22, 2023

Related U.S. Application Data
(60) Provisional application No. 63/344,423, filed on May 20, 2022.

(51) Int. Cl. A61F 13/04

(52) U.S. Cl. CPC A61F 13/046

(57) ABSTRACT

Disclosed is a ventilation system for a portion of person's skin covered by an enclosure that impedes the dissipation of body heat. The ventilation system comprises a pressurized air device and a ventilation apparatus. the pressurized air device is fluidly connected to the ventilation apparatus. The ventilation apparatus comprises a discharge suitable for delivery of pressurized air to the portion of skin covered by the enclosure.

### CROSS-REFERENCE TO RELATED APPLICATIONS

This application claims priority of U.S. Provisional Patent Application Ser. No. 63/344,423, entitled “COOLING SYSTEM,” filed on May 20, 2022, the contents of which are incorporated by reference herein in their entirety.

### FIELD OF THE INVENTION

This disclosure relates generally to personal ventilation systems. In particular, this disclosure relates to ventilation systems that provide cooling for portions of a person's body covered by an enclosure.

### BACKGROUND OF THE INVENTION

There is frequently a need for at least a portion of a person's body to be covered by an enclosure, such as, but not limited to, an orthotic, a cast, and a bed covering. Such enclosures often result in limited or no ventilation and/or cooling. Removal of different types of enclosures may be inconvenient or impossible, resulting in a limited ventilation to and/or a limited heat dissipation from the area of skin covered by the enclosure.

There is a need to provide systems and methods for providing ventilation and/or cooling to portions of a person's body covered by enclosures. With respect to such covered areas of a person's body, such systems and methods would improve comfort and/or hygiene for users of such systems and methods. Ideally, such systems and methods would be implemented with commonly used equipment and familiar techniques and, further, would be portable to allow mobility of the user while such systems and methods are in use.

### SUMMARY OF THE INVENTION

Disclosed herein is a ventilation system for a portion of skin covered by an enclosure, wherein the enclosure impedes the dissipation of body heat. The ventilation system comprises a pressurized air device and a ventilation apparatus. The pressurized air device is fluidly connected to the ventilation apparatus. The ventilation apparatus comprises a discharge suitable for delivery of pressurized air to the portion of skin covered by the enclosure. In some embodiments, the ventilation system further comprises a support device suited for attaching the pressurized air device to the enclosure.

In some embodiments, the pressurized air device comprises a compressor apparatus having an inlet suitable for drawing ambient air into the compressor apparatus.

In some embodiments, the pressurized air device comprises a pressurized air canister and a valve suitable for controlling the flow rate of pressurized air delivered to the ventilation apparatus.

In some embodiments, the enclosure comprises an orthosis, a cast, an article of clothing, or a layer of bedding.

The foregoing has outlined rather broadly the features and technical advantages of the present invention in order that the detailed description of the invention that follows may be better understood. Additional features and advantages of the invention will be described hereinafter, which form the subject matter of the claims of the invention. It should be appreciated by those skilled in the art that the conception and specific embodiments disclosed may be readily utilized as a basis for modifying or designing other apparatuses and/or methods for carrying out the same purposes of the present invention. It should also be realized by those skilled in the art that such equivalent constructions do not depart from the spirit and scope of the invention as set forth in the appended claims. The novel features which are believed to be characteristic of the invention, both as to its compositions and methods, together with further objects and advantages will be better understood from the following description.

### BRIEF DESCRIPTION OF THE FIGURES

The claimed subject matter may be understood by reference to the following description taken in conjunction with the accompanying drawings, in which like reference numerals identify like elements, and in which:

FIG. 1 is a view perspective view of a ventilation system according to an embodiment of the invention.

The drawing is not necessarily to scale and certain features may be shown exaggerated in scale or in somewhat schematic form in the interest of clarity and conciseness.

### DETAILED DESCRIPTION OF THE INVENTION

Illustrative embodiments of the subject matter claimed below will now be disclosed. In the interest of clarity, some features of some actual implementations may not be described in this specification. It will be appreciated that in the development of any such actual embodiments, numerous implementation-specific decisions must be made to achieve the developer's specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

### Definitions

As used herein, “a” or “an” when used in conjunction with the term “comprising” in the claims or the specification means one or more than one, unless the context dictates otherwise.

As used herein, “comprise”, “have”, “include” and “contain” (and their variants) are open-ended linking verbs and allow the addition of other elements when used in a claim.

As used herein, “consisting essentially of” excludes additional material elements, but allows the inclusions of non-material elements that do not substantially change the nature of the invention.

As used herein, “consisting of” is closed and excludes all additional elements.

As used herein, “functional brace” means an orthosis designed for use by people who have experienced an injury and need support to recover from it. Functional braces are also utilized to help people who suffer from pain associated with arthritis. Functional braces are intended to reduce the rotation of a joint, reduce the chance of hyperextension, stabilize and strengthen the joint, and generally increase agility and support stability of the wearer. The majority of functional braces are made of elastic.

As used herein, “knee brace” (KO) means an orthosis that extends above and below the knee joint and is generally worn to support or align the knee. In the case of diseases causing neurological or muscular impairment of muscles surrounding the knee, a KO can prevent flexion or extension instability of the knee. In the case of conditions affecting the ligaments or cartilage of the knee, a KO can provide stabilization to the knee by replacing the function of these injured or damaged parts. For instance, knee braces can be used to relieve pressure from the part of the knee joint affected by diseases such as arthritis or osteoarthritis by realigning the knee joint into valgus or varus. When used properly, a knee brace may help an individual to stay active by enhancing the position and movement of the knee or reducing pain.

As used herein, “lower-limb orthosis” or “lower-extremity orthosis” mean orthoses comprising mechanical or electromechanical devices applied externally to the ankle, knee, leg, or segments thereof in order to restore or improve function or structural characteristics of the segments encumbered by the device. In general, musculoskeletal problems that may be alleviated by the use of lower-limb orthoses include those resulting from trauma or disease (e.g., arthritis). Such orthoses may also be beneficial in aiding individuals who have a neurological impairment such as stroke, spinal cord injury, or peripheral neuropathy. Types of lower-limb orthoses include ankle-foot orthoses and knee orthoses. An ankle-foot orthoses is generally used to immobilize the ankle and lower leg in the presence of arthritis or fracture.

As used herein, “orthosis” or “orthotic” means a brace or other such device used in the field of medicine related to treatment and/or correction of disorders of the limbs or spine by use of braces and other devices to correct alignment or provide support. Orthosis as used herein refers broadly to a functional brace, a knee brace, a lower-limb orthosis, a rehabilitation brace, a relief orthosis, or an upper-limb orthosis.

As used herein, “pressurized air device” means an airflow device interfaces with an end of a ventilation tube to connect the airflow from the airflow device to the ventilation tubes. The airflow device can be any device that is capable of providing a flow of air to the ventilation tubes sufficient to cool the skin temperature of skin covered by an enclosure, such as, but not limited to an orthosis. The airflow device interfaces with the support device to affix or attach the airflow device to the relevant enclosure, such as, but not limited to an orthosis.

As used herein, “rehabilitation brace” means an orthosis used to limit the movement of the knee or elbow in both medial and lateral directions. These braces often have an adjustable range of motion for limiting flexion and extension following knee or elbow reconstruction. Rehabilitation braces are primarily used after injury or surgery to immobilize the leg or arm. Rehabilitation braces are generally larger in size than other braces, due to their function.

As used herein, “relief orthosis” means an orthosis used to control, guide, limit, and/or immobilize an extremity, joint, or body segment for a particular reason. In some embodiments, relief orthoses restrict movement in a given direction, assist movement generally, reduce weight-bearing forces for a particular purpose, aid rehabilitation from fractures after the removal of a cast, and otherwise correct the shape and/or function of the body to provide easier movement capability and/or reduction of pain.

As used herein, “upper-limb orthosis” or “upper extremity orthosis” mean orthoses are mechanical or electromechanical devices applied externally to the arm, or segments thereof in order to restore or improve function or structural characteristics of the arm segments encumbered by the device. In general, musculoskeletal problems that may be alleviated by the use of upper limb orthoses include those resulting from trauma or disease (e.g., arthritis). They may also be beneficial in aiding individuals who have a neurological impairment such as stroke, spinal cord injury, or peripheral neuropathy. The general types of upper-limb orthoses include upper-limb orthoses, clavicular and shoulder orthoses, arm orthoses, functional arm orthoses, elbow orthoses, forearm-wrist orthoses, forearm-wrist-thumb orthoses, forearm-wrist-hand orthoses, hand orthoses, upper-extremity orthoses (with special functions), or any other type of orthoses that is suitable for use above the waist of the patient.

As used herein, “ventilation apparatus” means any apparatus having an inlet suitable for fluidly connecting to the discharge of the pressurized air device and having one or more discharge holes or vents suitable for distributing the pressurized air between the inner surface of an enclosure and a portion of a person's skin covered by such enclosure. An example of such an enclosure is an orthosis.

### Ventilation System

Embodiments disclosed herein provide a personal ventilation system. In particular, embodiments disclosed herein provide a cooling system that can be integrated with or in conjuncture with an enclosure around a portion of a user's body, such as an orthosis, to provide a flow rate of air resulting in cooling the interior of the orthoses and the skin of the patient.

Disclosed herein is a ventilation system for a portion of skin covered by an enclosure. Such an enclosure impedes the normal dissipation of body heat. The ventilation system comprises a pressurized air device and a ventilation apparatus. The pressurized air device is fluidly connected to the ventilation apparatus. The ventilation apparatus comprises a discharge suitable for delivery of pressurized air to the portion of skin covered by the enclosure.

In some embodiments, the ventilation system further comprises a support device suited for attaching the pressurized air device to the enclosure. In some embodiments, the support device provides means of attaching or affixing the airflow device to the enclosure and/or the ventilation apparatus comprises a strap (optionally with a buckle), a clip, hook and loop fabric (e.g., a Velcro™ fastener), or a combination thereof. The support device can be integrated with the pressurized air device or it can be a separate component from the airflow device. The support device can be removed from one enclosure (e.g., an orthosis) and reused on another enclosure (e.g., and orthosis). The support device can be specific to a particular enclosure, a specific class of enclosures, or it can be universally applicable to all enclosures. The support device can be specific to a particular orthosis, a specific class of orthoses, or it can be universally applicable to all orthoses.

In some embodiments, the pressurized air device comprises a compressor apparatus having an inlet suitable for drawing ambient air into the compressor apparatus. In further embodiments, the compressor apparatus comprises a control system for controlling the pressure and/or flow rate of compressed air from the compressor apparatus into the ventilation apparatus. In some embodiments, the control system is manually operated by the user of the ventilation system. In operation, the patient selects the level of cooling that is desired from the airflow device, the airflow device then provides an amount of airflow related to the selected cooling setting to the ventilation tubes. The ventilation tubes then distribute the airflow throughout the enclosure, such as, but not limited to, an orthosis. The distributed airflow exits the cooling tubes through the cooling vents and onto the skin of the person covered by the enclosure, thereby cooling the skin covered by the enclosure, such as, but not limited to, an orthosis. The airflow device can be controlled by adjusting physical settings on the airflow device or the airflow device can be a controlled remotely via a wireless connection, such as a bluetooth connection to a patient's electronic device. The airflow device can be controlled by the patient to increase the airflow or decrease the airflow through the tubing to a suitable level as desired by the patient.

In some embodiments, the control system further comprises a temperature sensor suitable for disposition proximate to and detection of a temperature of the skin covered by the enclosure. Such control systems are operable to control the pressure and/or flow rate of compressed air delivered to the ventilation apparatus to maintain the detected temperature within a selected temperature range.

In some embodiments, the pressurized air device comprises a pressurized air canister and a valve suitable for controlling the flow rate of pressurized air delivered to the ventilation apparatus. In further embodiments, the ventilation system further comprises a control system for controlling the flow rate of pressurized air from the pressurized air canister into the ventilation apparatus. In addition to the air flow provided by the withdrawal of air from a pressurized canister, the pressure drop and expansion of air exiting the canister provides additional cooling. Other aspects of the control system for controlling the pressurized air canister can be the same as for the compressor. In some embodiments, the control system is manually operated by the user of the ventilation system. In some embodiments, the control system further comprises a temperature sensor suitable for disposition proximate to and detection of a temperature of the skin covered by the enclosure. Such control systems are operable to control is operable to control the flow rate of pressurized air delivered to the ventilation apparatus to maintain the detected temperature within a selected temperature range.

In some embodiments of the ventilation system, the enclosure comprises an orthosis and the portion of skin covered corresponds to an inner surface of the orthosis. Orthoses that are commonly available do not provide adequate ventilation to the portion of the body that is covered by the orthotic, which ultimately can cause discomfort and excessive sweating by the patient. By controlling the sweat with a ventilation system, the patient can ambulate normally with reduced discomfort. The system can be used by the patient during general everyday activities such as walking, resting, or physical therapy sessions. Additionally, the pressurized air device will provide a cooling comfort throughout the orthosis. The ventilation system aids with providing a circulation of air and/or cooling down the area of the patient's body covered by the orthosis to reduce or eliminate perspiration. The ventilation system further aids in diminishing the discomfort associated with excess body heat trapped within the orthosis. The ventilation system can be controlled to match the subjective preferences of each individual patient.

Patients generally use orthoses after injuries to joints to stabilize the injured joint during recovery. These include torn ligaments and wear and tear of joints. Orthoses are also used after operations such as a complete replacement of a joint or operations on the joint structure or other operations on bony, muscular structures or operations on the joint ligaments. Orthoses that are commonly available do not provide adequate ventilation to the portion of the body that is covered by the orthotic, which ultimately can cause discomfort and excessive sweating by the patient. For example, the cooling system may be implemented with an ankle-foot orthoses to reduce the temperature inside the orthoses and reduce the overall amount of perspiration of a patient utilizing the ankle-foot orthoses.

In some embodiments suited for use with an orthosis, the ventilation apparatus comprises one or more tubes, wherein each tube has an inlet for receiving air from the pressurized air device, is closed at the end opposite the inlet, and has holes disposed along a length of the tube. In some embodiments, one or more tubes are inserted in between the patient's skin and the inner surface of the orthosis after the patient has donned the orthosis. In some embodiments, one or more tubes are inserted into the orthosis prior to the patient donning the orthosis, such that the tubes will be between the patient's skin and the inner surface of the orthosis after the patient has donned the orthosis. In some embodiments, the tubes are an integral part of the orthosis.

In some embodiments of the ventilation system, the enclosure comprises a cast and the portion of skin covered corresponds to an inner surface of the cast. In some embodiments, the ventilation apparatus comprises one or more tubes, wherein each tube has an inlet for receiving air from the pressurized air device, is closed at the end opposite the inlet, and has holes disposed along a length of the tube.

In some embodiments of the ventilation system, the enclosure comprises an article of clothing and the portion of skin covered corresponds to an inner surface of the article of clothing, such as, but not limited to, a shoe, a boot, or a snow skiing coverall. In some embodiments, the ventilation apparatus comprises one or more tubes, wherein each tube has an inlet for receiving air from the pressurized air device, is closed at the end opposite the inlet, and has holes disposed along a length of the tube. In further embodiments, the one or more tubes are supported on an undergarment corresponding to the article of clothing.

In some embodiments of the ventilation system, the enclosure comprises an upper surface of a bed and at least one layer of bedding and the portion of skin covered corresponds to a portion of a body enclosed by a portion of the upper surface of a bed and a portion of the at least one layer of bedding. In some embodiments, the ventilation apparatus comprises one or more tubes, wherein each tube has an inlet for receiving air from the pressurized air device, is closed at the end opposite the inlet, and has holes disposed along a length of the tube. In further embodiments, the one or more tubes are supported on a second bedding layer corresponding to the at least one bedding layer.

### Certain Embodiments

In a first set of embodiments, a ventilation system for a portion of skin covered by an enclosure comprises a pressurized air device and a ventilation apparatus. The pressurized air device is fluidly connected to the ventilation apparatus. The ventilation apparatus comprises a discharge suitable for delivery of pressurized air to the portion of skin covered by the enclosure. In some embodiments, the ventilation system further comprises a support device suited for attaching the pressurized air device to the enclosure. In some embodiments, the support device provides means of attaching or affixing the airflow device to the enclosure and/or the ventilation apparatus comprises a strap (optionally with a buckle), a clip, hook and loop fabric, or a combination thereof.

In a second set of embodiments, in addition to one or more limitations of the first set of embodiments, the pressurized air device comprises a compressor apparatus having an inlet suitable for drawing ambient air into the compressor apparatus. In further embodiments, the compressor apparatus comprises a control system for controlling the pressure and/or flow rate of compressed air from the compressor apparatus into the ventilation apparatus. In some embodiments, the control system is manually operated by the user of the ventilation system. In some embodiments, the control system further comprises a temperature sensor suitable for disposition proximate to and detection of a temperature of the skin covered by the enclosure. Such control systems are operable to control the pressure and/or flow rate of compressed air delivered to the ventilation apparatus to maintain the detected temperature within a selected temperature range.

In a third set of embodiments, in addition to one or more limitations of the first set of embodiments, the pressurized air device comprises a pressurized air canister and a valve suitable for controlling the flow rate of pressurized air delivered to the ventilation apparatus. In further embodiments, the ventilation system further comprises a control system for controlling the flow rate of pressurized air from the pressurized air canister into the ventilation apparatus. In some embodiments, the control system is manually operated by the user of the ventilation system. In some embodiments, the control system further comprises a temperature sensor suitable for disposition proximate to and detection of a temperature of the skin covered by the enclosure. Such control systems are operable to control is operable to control the flow rate of pressurized air delivered to the ventilation apparatus to maintain the detected temperature within a selected temperature range.

In a fourth set of embodiments, in addition to one or more limitations of the first and second sets of embodiments, or alternatively, in addition to one or more limitations of the first and third sets of embodiments, the enclosure comprises an orthosis and the portion of skin covered corresponds to an inner surface of the orthosis. In some embodiments, the ventilation apparatus comprises one or more tubes, wherein each tube has an inlet for receiving air from the pressurized air device, is closed at the end opposite the inlet, and has holes disposed along a length of the tube.

In a fifth set of embodiments, in addition to one or more limitations of the first and second sets of embodiments, or alternatively, in addition to one or more limitations of the first and third sets of embodiments, the enclosure comprises a cast and the portion of skin covered corresponds to an inner surface of the cast. In some embodiments, the ventilation apparatus comprises one or more tubes, wherein each tube has an inlet for receiving air from the pressurized air device, is closed at the end opposite the inlet, and has holes disposed along a length of the tube.

In a sixth set of embodiments, in addition to one or more limitations of the first and second sets of embodiments, or alternatively, in addition to one or more limitations of the first and third sets of embodiments, the enclosure comprises an article of clothing and the portion of skin covered corresponds to an inner surface of the article of clothing. In some embodiments, the ventilation apparatus comprises one or more tubes, wherein each tube has an inlet for receiving air from the pressurized air device, is closed at the end opposite the inlet, and has holes disposed along a length of the tube. In further embodiments, the one or more tubes are supported on an undergarment corresponding to the article of clothing.

In a seventh set of embodiments, in addition to one or more limitations of the first and second sets of embodiments, or alternatively, in addition to one or more limitations of the first and third sets of embodiments, the enclosure comprises an upper surface of a bed and at least one layer of bedding and the portion of skin covered corresponds to a portion of a body enclosed by a portion of the upper surface of a bed and a portion of the at least one layer of bedding. In some embodiments, the ventilation apparatus comprises one or more tubes, wherein each tube has an inlet for receiving air from the pressurized air device, is closed at the end opposite the inlet, and has holes disposed along a length of the tube. In further embodiments, the one or more tubes are supported on a second bedding layer corresponding to the at least one bedding layer.

### EXAMPLES

### Example 1

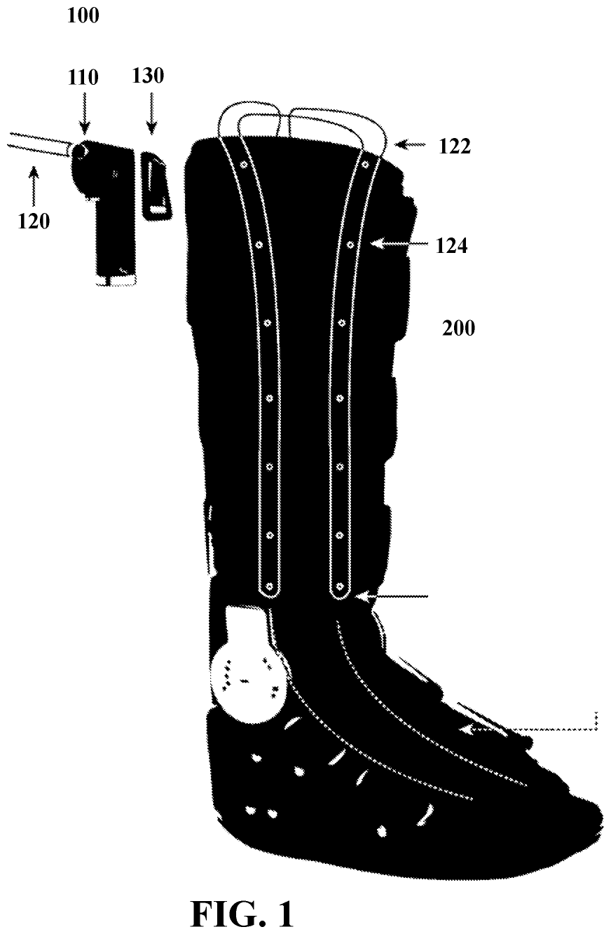

FIG. 1 shows an embodiment of a ventilation system 100 for use in conjunction with an orthoses 200. The ventilation system 100 (all components other than orthoses 200) comprises a pressurized air device comprising a compressor 110, a ventilation apparatus comprising tubes 120,122, and a support device comprising a clip 130. Clip 130 could be permanently or detachably attached to the compressor 110. The discharge of the compressor 110 would be attached to the inlet tube 120, which in turn would be connected to inlets of tubes 122 through a manifold (not shown). Note that the ends of tubes 122 opposite their respective inlets are capped to force pressurized air inside tubes 122 through holes 124 to provide distribution of pressurized air between the interior of the orthosis 200 and the skin of a patient wearing the orthosis. In operation, the patient selects the level of cooling that is desired from the compressor 110. The compressor 110 then provides an amount of airflow related to the selected cooling setting to the tubes 122. The tubes 122 then distribute the airflow throughout the orthoses 200. The distributed airflow exits the tubes 122 through the holes 124 and onto the skin of the patient, thereby cooling the area covered by the orthoses 200.

The cooling tubes 122 can be installed in the orthosis 200 by the patient, an assistant to the patient, or by a medical professional. The cooling tubes 122 can be installed into orthosis 200 before or after the patient dons the orthosis 200. The cooling tubes can be made of any flexible material that is compatible with both the temperatures of the interior of the orthoses when the cooling airflow is not in use and the expected temperatures of the cooling tubes when the airflow is in flowing through the tubes. In the embodiment shown in FIG. 1, the tubes 122 extend the full length of the orthosis 200.

The cooling tubes are provided with a series of holes or vents to allow the airflow to exit the tubes and ventilation within the orthosis 200 to cool the skin of the patient. The tubes 122 can be affixed to the interior of the orthoses 200 by the patient.

Although embodiments of the ventilation system have been described in detail, those skilled in the art will also recognize that various substitutions and modifications may be made without departing from the scope and spirit of the appended claims.

In the foregoing description of certain embodiments, specific terminology has been resorted to for the sake of clarity. However, the disclosure is not intended to be limited to the specific terms so selected, and it is to be understood that each specific term includes other technical equivalents which operate in a similar manner to accomplish a similar technical purpose. Terms such as “left” and right”, “front” and “rear”, “above” and “below” and the like are used as words of convenience to provide reference points and are not to be construed as limiting terms.

In addition, the foregoing describes some embodiments of the disclosure, and alterations, modifications, additions and/or changes can be made thereto without departing from the scope and spirit of the disclosed embodiments, the embodiments being illustrative and not restrictive.

Furthermore, the disclosure is not to be limited to the illustrated implementations, but to the contrary, is intended to cover various modifications and equivalent arrangements included within the spirit and scope of the disclosure. Also, the various embodiments described above may be implemented in conjunction with other embodiments, e.g., aspects of one embodiment may be combined with aspects of another embodiment to realize yet other embodiments. Further, each independent feature or component of any given assembly may constitute an additional embodiment.

What is claimed is:

1. A ventilation system for a portion of skin covered by an enclosure that impedes the dissipation of body heat, the ventilation system comprising:
a pressurized air device; and
a ventilation apparatus;
wherein:
the pressurized air device is fluidly connected to the ventilation apparatus; and
the ventilation apparatus comprises a discharge suitable for delivery of pressurized air to the portion of skin covered by the enclosure.

2. The ventilation system of claim 1, further comprising a support device suited for attaching the pressurized air device to the enclosure.

3. The ventilation system of claim 1, wherein the pressurized air device comprises a compressor apparatus having an inlet suitable for drawing ambient air into the compressor apparatus.

4. The ventilation system of claim 2, wherein the compressor apparatus comprises a control system for controlling the pressure and/or flow rate of compressed air from the compressor apparatus into the ventilation apparatus.

5. The ventilation system of claim 4, wherein the control system is manually operated.

6. The ventilation system of claim 4, wherein the control system:
further comprises a temperature sensor suitable for disposition proximate to and detection of a temperature of the skin covered by the enclosure; and
is operable to control the pressure and/or flow rate of compressed air delivered to the ventilation apparatus to maintain the detected temperature within a selected temperature range.

7. The ventilation system of claim 1, wherein the pressurized air device comprises a pressurized air canister and a valve suitable for controlling the flow rate of pressurized air delivered to the ventilation apparatus.

8. The ventilation system of claim 7, wherein the ventilation system further comprises a control system for controlling the flow rate of pressurized air from the pressurized air canister into the ventilation apparatus.

9. The ventilation system of claim 8, wherein the control system is manually operated.

10. The ventilation system of claim 8, wherein the control system:
further comprises a temperature sensor suitable for disposition proximate to and detection of a temperature of the skin covered by the enclosure; and
is operable to control the flow rate of pressurized air delivered to the ventilation apparatus to maintain the detected temperature within a selected temperature range.

11. The ventilation system of claim 1, wherein the enclosure comprises an orthosis and the portion of skin covered corresponds to an inner surface of the orthosis.

12. The ventilation system of claim 11, wherein the ventilation apparatus comprises one or more tubes, wherein each tube:
has an inlet for receiving air from the pressurized air device;
is closed at the end opposite the inlet; and
has holes disposed along a length of the tube.

13. The ventilation system of claim 1, wherein the enclosure comprises a cast and the portion of skin covered corresponds to an inner surface of the cast.

14. The ventilation system of claim 13, wherein the ventilation apparatus comprises one or more tubes, wherein each tube:
has an inlet for receiving air from the pressurized air device;
is closed at the end opposite the inlet; and
has holes disposed along a length of the tube.

15. The ventilation system of claim 1, wherein the enclosure comprises an article of clothing and the portion of skin covered corresponds to an inner surface of the article of clothing.

16. The ventilation system of claim 15, wherein the ventilation apparatus comprises one or more tubes, wherein each tube:
has an inlet for receiving air from the pressurized air device;
is closed at the end opposite the inlet; and
has holes disposed along a length of the tube.

17. The ventilation system of claim 16, wherein the one or more tubes are supported on an undergarment corresponding to the article of clothing.

18. The ventilation system of claim 1, wherein the enclosure comprises an upper surface of a bed and at least one layer of bedding and the portion of skin covered corresponds to a portion of a body enclosed by a portion of the upper surface of a bed and a portion of the at least one layer of bedding.

19. The ventilation system of claim 18, wherein the ventilation apparatus comprises one or more tubes, wherein each tube:
has an inlet for receiving air from the pressurized air device;
is closed at the end opposite the inlet; and
has holes disposed along a length of the tube.

20. The ventilation system of claim 19, wherein the one or more tubes are supported on an a second bedding layer corresponding to the at least one bedding layer.
